(19) United States
(12) Patent Application Publication
(10) Pub. No.: US 2008/0318309 A1
(43) Pub. Date: Dec. 25, 2008

(54) SUBMERGED MEMBRANE BIO-REACTOR

(75) Inventors: In Soung Chang, Chungcheongnam-do (KR); Dong Hwan Shin, Chungcheongnam-do (KR); Byung Do Baek, Seoul (KR)

Correspondence Address: LARSON AND LARSON, 11199 69TH STREET NORTH, LARGO, FL, 33773, US

(73) Assignee: HOSEO UNIVERSITY ACADEMIC COOPERATION FOUNDATION, Chungcheongnam-do (KR)

(21) Appl. No.: 11/966,352

(22) Filed: Dec. 28, 2007

(30) Foreign Application Priority Data
Jun. 20, 2007 (KR) 10-2007-0060293

(51) Int. Cl. C12M 1/24

(52) U.S. Cl. 435/304.1

(57) ABSTRACT

A submerged membrane bio-reactor (MBR) is provided. The submerged MBR includes a submerged membrane module having a membrane with a hollow fiber structure, a cylindrical tube covering an outer circumference of the submerged membrane module, and a nozzle or a porous diffuser provided within a treatment vessel to supply air to inside of the cylindrical tube. Air bubbles generated from the nozzle or the porous diffuser flows into the cylindrical tube in a slug-type liquid flow that effectively decreases membrane contamination.

### BACKGROUND OF THE INVENTION

1. Field of the Invention

The present invention relates to a membrane technology for sewage and wastewater treatment, and more particularly, to a submerged membrane bio-reactor (MBR) having a submerged membrane module and a cylindrical tube that covers the outer circumference of the submerged membrane module.

2. Description of the Prior Art

Membrane technology which has been used for sewage and wastewater treatment for the last 20 years is gradually expanding in its practical application and has been receiving attention as a reliable technology for advanced treatment of reusable sewage and wastewater. As one of such a technology, a membrane bio-reactor (MBR) technology combines advantages of the membrane technology and activated sludge processing technology to replace and overcome the drawbacks of conventional sedimentation methods of treating a large amount of activated sludge. The MBR technology is also referred to as an activated sludge membrane separation process or an activated sludge multi-membrane separation process, which can be implemented with a bio-reactor to form a membrane bio-reactor. Particularly, the use of submerged membrane bio-reactors for sewage and wastewater treatment is increasing since they can be used for a high-capacity treatment having thorough filtration results to obtain a stable amount of reusable water.

Despite the advantages in treating sewage and wastewater, the wide use of MBRs is being hindered due to the membrane fouling problem. That is, there is a problem of decreased water yield (flux) caused by the accumulation of cake layer on the membrane surface. In order to obtain a stable water yield or maintain a constant flux, contaminated membranes must be frequently cleaned through a chemical/physical cleaning process or the membranes must be replaced regularly, which increases the cost of operating and maintaining the submerged MBRs.

Membrane contamination is commonly indicated by membrane resistance which may be reversible in most cases or irreversible in some cases. Here, irreversible membrane resistance is due to the clogging of micropores in the membrane, while reversible membrane resistance is due to the accumulation of cake layer on the membrane surface over a period of time. In order to control the reversible membrane resistance, a method of supplying air to generate shear force around the membrane surface to impede the accumulation of cake layer is commonly implemented. The air provided to control membrane contamination is also used to process microbes in the activated sludge in submerged MBRs.

Thus, an excess amount of air is needed beyond the amount needed to prevent the accumulation of the activated sludge on the membrane surface. As a result, there is a problem of increasing the operating cost of supplying the air, which outweighs the benefits obtained from preventing membrane contamination. Therefore, there is a need to optimize the use of air provided to the membrane surface to obtain a maximum cleaning efficiency from the air provided in MBRs.

### SUMMARY OF THE INVENTION

The present invention is to decrease the resistance of a cake layer which causes the contamination of membrane and decreases flux during the operation of a membrane bio-reactor (MBR) or a submerged MBR.

Accordingly, an aspect of the present invention provides a submerged MBR including a submerged membrane module and a cylindrical tube covering the submerged membrane module, in which air supplied to the cylindrical tube is prevented from being escaped to maximize the cleaning efficiency of the air.

Another aspect of the present invention provides a submerged MBR, in which the contamination of a membrane can be more effectively prevented than a conventional method of using a porous diffuser, when air is supplied at the same flow rate.

Another aspect of the present invention provides a submerged MBR, in which air supplied from a nozzle or a porous diffuser to a cylindrical tube induces in an effective two-phase flow of liquid and air bubbles.

However, aspects of the present invention are not restricted to the one set forth herein. The above and other aspects of the present invention will become more apparent to one of ordinary skill in the art to which the present invention pertains by referencing the detailed description of the present invention given below.

According to an aspect of the present invention, there is provided a submerged MBR including: a submerged membrane module having a membrane with a hollow fiber structure; a cylindrical tube covering an outer circumference of the submerged membrane module; and a nozzle or a porous diffuser provided within a treatment vessel to supply air to inside of the cylindrical tube.

According to another aspect of the present invention, there is provided a submerged MBR including: a submerged membrane module having a membrane with a hollow fiber structure; a cylindrical tube covering an outer circumference of the submerged membrane module; and a nozzle provided within a treatment vessel to supply air to inside of the cylindrical tube, wherein a cross-sectional area ratio (Aₘ/Aₜ) of the membrane to the cylindrical tube is from about 0.50 to about 0.60, Aₘ being a cross-sectional area of the membrane of the submerged membrane module, Aₜ being a cross-sectional area of the cylindrical tube.

According to another aspect of the present invention, there is provided a submerged MBR including: a submerged membrane module having a membrane with a hollow fiber structure; a cylindrical tube covering an outer circumference of the submerged membrane module; and a porous diffuser provided within a treatment vessel to supply air to inside of the cylindrical tube, wherein a cross-sectional area ratio (Aₘ/Aₜ) of the membrane to the cylindrical tube is from about 0.25 to about 0.30, Aₘ being a cross-sectional area of the membrane in the submerged membrane module, Aₜ being a cross-sectional area of the cylindrical tube.

### BRIEF DESCRIPTION OF THE DRAWINGS

The above and other aspects and features of the present invention will become more apparent by describing in detail exemplary embodiments thereof with reference to the attached drawings, in which:

FIG. 1 is a mimetic diagram illustrating a submerged MBR according to an exemplary embodiment of the present invention;

FIG. 2 illustrates a cross-sectional view of a membrane module arranged with a cylindrical tube, according to an exemplary embodiment of the present invention;

FIG. 3 is a mimetic diagram illustrating change in liquid flow inside of a cylindrical tube with increasing air bubbles, according to an exemplary embodiment of the present invention;

FIGS. 4A and 4B are graphs respectively illustrating change in trans-membrane pressure (TMP) and change in total resistance (Rₜ)/intrinsic membrane resistance (Rₘ) with respect to time when a nozzle and a porous diffuser, according to exemplary embodiments of the present invention, are respectively operated with different air flow rates;

FIG. 5A is a graph illustrating change in Rₜ/Rₘ with respect to time when a nozzle and a porous diffuser, according to exemplary embodiments of the present invention, are respectively operated with different fluxes (outflow generating rate); and

FIGS. 6A and 6B are graphs illustrating change in Rₜ/Rₘ with respect to time when a nozzle and a porous diffuser, according to exemplary embodiments of the present invention, are respectively operated with different membrane sizes.

### DETAILED DESCRIPTION OF THE PREFERRED EMBODIMENT

The present invention will now be described more fully with reference to the accompanying drawings, in which exemplary embodiments of the invention are shown. The invention may, however, be embodied in many different forms and should not be construed as being limited to the embodiments set forth herein. Rather, these embodiments are provided so that this disclosure will be thorough and complete, and will fully convey the concept of the invention to those skilled in the art. Wherever possible, the same reference numerals will be used throughout the drawings to refer to the same or like parts.

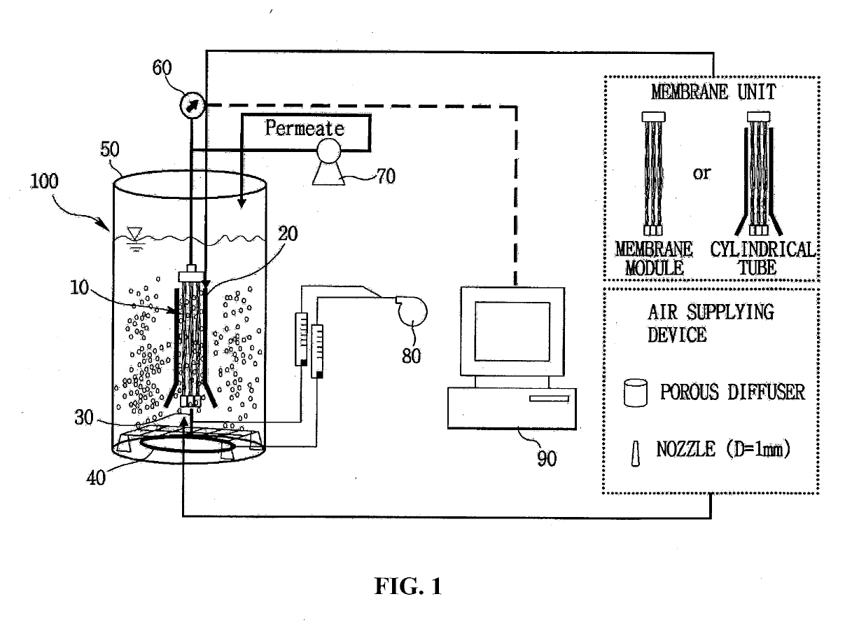

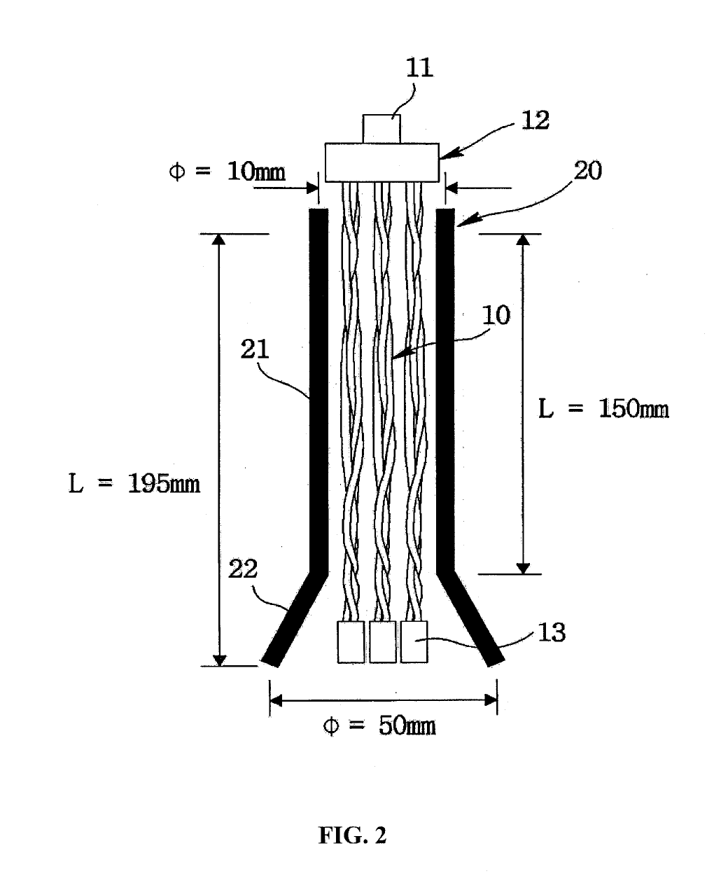

FIG. 1 is a mimetic diagram illustrating a submerged membrane bio-reactor (MBR) according to an exemplary embodiment of the present invention, and FIG. 2 illustrates a cross-sectional view of a membrane module arranged with a cylindrical tube, according to an exemplary embodiment of the present invention.

As shown in FIG. 1, the MBR includes a submerged membrane module 10 having a membrane with a hollow fiber structure, a cylindrical tube 20 covering an outer circumference of the submerged membrane module 10, and a nozzle 30 provided within a treatment vessel 50 to supply air to inside of the cylindrical tube 20.

Here, the membrane with a hollow fiber structure means a separator which is composed thread or fiber having holes in a center thereof. As shown in FIG. 1, the hole may be continuous or discontinuous in a center of a lengthwise direction of the membrane. In the present invention, in order to decrease the resistance of a cake layer which causes the contamination of membrane and decreases flux during the operation of the submerged MBR, the cylindrical tube 20 is provided to cover the membrane module 10 which optimizes the cleaning efficiency of air supplied to the treatment vessel (bio-reacting chamber) 50. That is, the cylindrical tube 20 prevents dispersion and dissipation of air bubbles in the treatment vessel 50 and prolongs the time that the air bubbles make contact with the surface of the hollow-fiber structured membrane of the membrane module 10. Further, the cylindrical tube 20 also makes it possible to realize a two-phase flow of liquid and air bubbles which is effective in cleaning the membrane to decrease contamination.

As shown in FIG. 2, the cylindrical tube 20, which covers the outer circumference of the submerged membrane module 10, has a cylindrical body 21 and a conical lower portion 22 formed on the cylindrical body 21. The conical lower portion 22 has a gradually increasing inside diameter larger than that of the cylindrical body 21. Here, the conical lower portion 22 enables the air and wastewater to easily enter the cylindrical tube 20 and prevents the air bubble and wastewater introduced into the membrane module 22 from being escaped or dispersed to outside.

According to the present invention, the air bubbles generated from the nozzle 30 flows into the cylindrical tube 20 in a slug-type liquid flow which prevents the contamination of the membrane in the membrane module 10 more effectively than the air bubbles generated from a porous diffuser 40, which will be described later in detail.

The nozzle 30 has a slender tube shape and is provided at a lower side of the cylindrical tube 20. Here, it is preferable that the nozzle 30 has a diameter of from about 0.1 mm to about 10 mm. If the nozzle 30 has a diameter greater or less than this range, a sufficient amount of the air cannot be provided to the cylindrical tube 20 or a slug-type liquid flow cannot be obtained. On the other hand, the porous diffuser 40 is a conventional disk-shaped porous diffuser which is comparable to the nozzle 30 in terms of injecting air through a plurality of its injection pipes.

Meanwhile, in the submerged membrane bio-reactor according to the present invention which includes the submerged membrane module 10, the cylindrical tube 20, and the nozzle 30 provided in the treatment vessel 50, a cross-sectional area ratio (Aₘ/Aₜ) of the membrane to the cylindrical tube 20 is from about 0.50 to about 0.60, Aₘ being a cross-sectional area of the membrane in the submerged membrane module 10, Aₜ being a cross-sectional area of the cylindrical tube 20.

On the other hand, when the porous diffuser 40 is provided within the treatment vessel 50 instead of the nozzle 30, a cross-sectional area ratio (Aₘ/Aₜ) of the membrane to the cylindrical tube 20 is from about 0.25 to about 0.30, Aₘ being a cross-sectional area of the membrane in the submerged membrane module 10, Aₜ being a cross-sectional area of the cylindrical tube 20

Inventors of the present invention have used various methods of supplying air by using the nozzle 30 and/or the porous diffuser 40 below the lower portion of the cylindrical tube 20 covering the outer circumference of the membrane module 10 and have observed the effect this has on decreasing the contamination of the membrane of the module 10. Specifically, inventors of the present invention observed the presence of a two-phase flow of liquid and air bubbles by changing the amount of air supplied and the contamination of the membrane of the membrane module 10 by changing the operating flux and the number of membranes in the membrane module 10, and have obtained the following results.

When an insufficient amount of air is supplied from the nozzle 30, an activated sludge mixture quickly accumulated on the inner wall of the cylindrical tube 20 to rapidly clog the cylindrical tube 20, and, after a certain period of time, it was observed that the membrane of the submerged membrane module 10 contaminated more faster than as opposed to supplying air from the porous diffuser 40. From this observation, it was determined that the increase or decrease in membrane contamination was not due to increased or decreased number of membranes (increased/decreased Aₘ/Aₜ ratio) in the membrane module 10 covered by the cylindrical tube 20, rather there is an optimum Aₘ/Aₜ ratio which renders minimum membrane contamination.

That is, when the submerged MBR according to an exemplary embodiment of the present invention is provided with a porous diffuser as the air supply means, the optimum Aₘ/Aₜ ratio, which renders a maximum prevention of membrane contamination, is determined to be about 0.25 to about 0.30, and when provided with a nozzle as the air supply means, the optimum Aₘ/Aₜ ratio is determined to be about 0.50 to about 0.60. Here, the detailed description will be provided later.

Exemplary embodiments according to the present invention will be described in detail. In an exemplary embodiment of the present invention, the nozzle 30 or the porous diffuser 40 was provided below the cylindrical tube 20 covering the submerged membrane module 10. Thereafter, the degree of membrane contamination, according to change in the amount air supplied through the nozzle or the porous diffuser 40 and change in the cross-sectional area of the membrane in the submerged membrane module 10 covered by the cylindrical tube 20, was quantified. Additionally, the change in trans-membrane pressure (TMP) and the resistance of cake layer and clogged micropores were measured to quantify the effect of having the cylindrical tube 20.

### Experiment 1: Activated Sludge Growth

A mixed liquor suspended solids (MLSS) obtained from an environmental laboratory company (“C” City, South Choongnam Province) was acclimated with synthetic wastewater for 6 months. In the synthetic wastewater, glucose was used as carbon source and ammonium sulfate was used as nitrogen source. Composition and density thereof are shown in Table 1 below, and operating conditions for growing activated sludge are shown in Table 2.

TABLE 1
Composition and Density of Synthetic Wastewater

| Composition | Units | Density |
|---|---|---|
| Glucose | mg/L | 983.7 |
| Peptone | mg/L | 737.3 |
| Yeast extract | mg/L | 98.2 |
| (NH₄)₂SO₄ | mg/L | 830.0 |
| KH₂PO₄ | mg/L | 263.2 |
| MgSO₄—7H₂O | mg/L | 196.7 |
| MnSO₄—4H₂O | mg/L | 17.7 |
| FeCl₃—6H₂O | mg/L | 1.0 |
| CaCl₂—2H₂O | mg/L | 19.7 |
| NaHCO₃ | mg/L | 123-1230 |

TABLE 2
Operating Conditions for Growing Activated Sludge
Operating Condition

| Parameter | Units | Value |
|---|---|---|
| F/M ratio | gCOD/gMLSS | 0.20-0.25 |
| Hydraulic retention time | hour | 10-12 |
| Solids retention time | day | 20 |
| Aeration zone volume | L | 10 |
| Settling time | min | 30 |
| Air flow rate | L/min | 2.0 |
| pH | — | 7.0 ± 0.3 |
| Temperature | ° C. | 20 ± 3 |
| MLSS | mg/L | 6,000-6,500 |

### Experiment 2: Characteristics of MBR Having Submerged Cylindrical Tube and Operating Method Thereof

For the filtration test, a membrane having a hollow-fiber structure made of hydrophilic polyethylene (PE) reformed from its hydrophobic state was used as a microfiltration membrane having pore size of 0.4 μm. Characteristics and specifications of the membrane are shown in Table 3 below.

TABLE 3
Operating Conditions for Growing Activated Sludge

| | Characteristics |
|---|---|
| Type | Hollow fiber |
| Material | PE, polyethylene (hydrophilic) |
| Pore size | 0.4 μm |
| Outer diameter | 0.52 mm |
| Filtration mode | Out-In |
| Manufacturer | Mitsubishi Co., Japan |

Run 1, Run 2 and Run 3 were carried out by maintaining the flux introduced to the cylindrical tube in the MBR at 24 lm⁻¹h⁻¹. A membrane module having a total membrane surface area of 0.0034 m2 was used for Run 1, a membrane module having a total membrane surface area of 0.0051 m2 was used for Run 2, and a membrane module having a total membrane surface area of 0.0102 m2 was used for Run 3. The membrane modules respectively having 10, 15 and 30 membrane strands were used in the cylindrical tube for Run 1, Run 2, and Run 3, respectively. Since the total cross-sectional area of the membrane modules occupying in the cylindrical tube is different for each run, it is expected that the total area of the membrane strands passed by air bubbles is different for each run to affect the degree of membrane contamination. Further, the degree of membrane contamination was also observed by changing the operating flux from 24 to 35 lm⁻¹h⁻¹.

A transparent cylindrical acryl tube having an inside diameter of 10 mm and a length of 150 mm was used. The cylindrical tube was provided with a lower portion having a conical shape, a length of 45 mm, and a gradually increasing inside diameter of from 10 mm to 50 mm. Specifications of the membrane module and the cylindrical tube are shown in FIG. 1.

Before the experiment, 8 liters of activated sludge were dispersed in the acryl cylindrical tube. Then, a control membrane module without the cylindrical tube and the membrane modules (provided with the cylindrical tube) for Run 1, Run 2 and Run 3, respectively, were submerged in the treatment vessel shown in FIG. 1, and the MBR was operated for 600 minutes or until TMP reached 40 kPa using a peristaltic pump (Cole-Parmer Instrument Co., USA).

To mix the activated sludge and decrease membrane contamination, air was supplied at 1 liter/min using a porous diffuser having a ring-shape or a nozzle, both being provided on the bottom of the MBR. In order to determine the fluid characteristics of two-phase flow of liquid and air bubbles, the amount of activated sludge mixture (liquid) rising in the cylindrical tube was measured. The amount (mass) of liquid mixture escaping from the cylindrical tube was measured over a certain period time using an electronic scale (Sartorius LP220s, Germany) and transmitted to a computer, which was then converted to volume using the data from the computer to quantify the amount of liquid escaped from the cylindrical tube. The amount of air supplied to the cylindrical tube was adjusted to 0.5, 1.0, 1.5 and 2.0 liter/min using a flow meter. The manner in which the liquid and air bubbles flowed in two-phase was then adjusted based on the amount of liquid and air bubbles measured above.

### Experiment 3: Membrane Contamination and TMP Measurement

In order to quantify membrane contamination respect to change in the amount of air supplied and change in the area occupied by the membrane in the cylindrical tube, TMP was measured while maintaining constant flux. By using a digital pressure gauge (ZSE40F, SMC Co., Japan) mounted on the leading end of a peristaltic pump, TMP was measured and transmitted to a computer and change in TMP with respect to filtration time was observed to quantify membrane contamination.

Membrane contamination was quantified by using the measured TMP values and a resistance in series model to calculate the resistance from Equation (1) below:

$$J = \frac{T M P}{μ ( {R}_{T} )} = \frac{T M P}{μ ( {R}_{m} + {R}_{c} + {R}_{f} )} \qquad ( 1 )$$

where, J=flux, μ=viscosity of permeate, R_{T}=total resistance, Rₘ=intrinsic membrane resistance, R_{c}=cake layer resistance, and R_{f}=pore fouling resistance.

Prior to filtering the activated sludge, intrinsic membrane resistance Rₘ was obtained by using the TMP value measured for filtering deionized water. When TMP has reached 40 kPa due to the accumulation of the activated sludge on the membrane surface or when 600 minutes has elapsed at which the peristaltic pump was stopped, total resistance R_{T} was obtained by using the measured TMP value. Then, after removing the cake layer accumulated on the membrane surface, pore fouling resistance R_{f} was calculated by using data obtained by filtering deionized water. To obtain the resistance values, the MBR was operated with the conditions shown in Table 4 below.

TABLE 4
MBR Operating Conditions

| Parameter | Range |
|---|---|
| Permeate flux (L/m² · hr) | 24/35 |
| HRT (h) | 10-12 |
| SRT (day) | 25-30 |
| MLSS (mg/L) | 6,000-6,500 |
| Temperature (° C.) | 20 ± 3 |

### Example 1

### Analysis of Two-Phase Flow in Cylindrical Tube

By increasing the amount of air supplied by a porous diffuser and a nozzle in steps of 0.5, 1.0, 1.5, and 2.0 L/min, amount Q, of the activated sludge liquid mixture escaped outside of the cylindrical tube was observed, and the measured amount Q, of the activated sludge liquid mixture did not show significant difference between the nozzle and the porous diffuser. Then, after measuring amounts Qg of air bubbles and Q, of liquid, ratio of the amount of air bubbles to liquid ε was calculated from Equation (2) below.

$$ɛ = \frac{{Q}_{g}}{{Q}_{g} + {Q}_{l}} \qquad ( 2 )$$

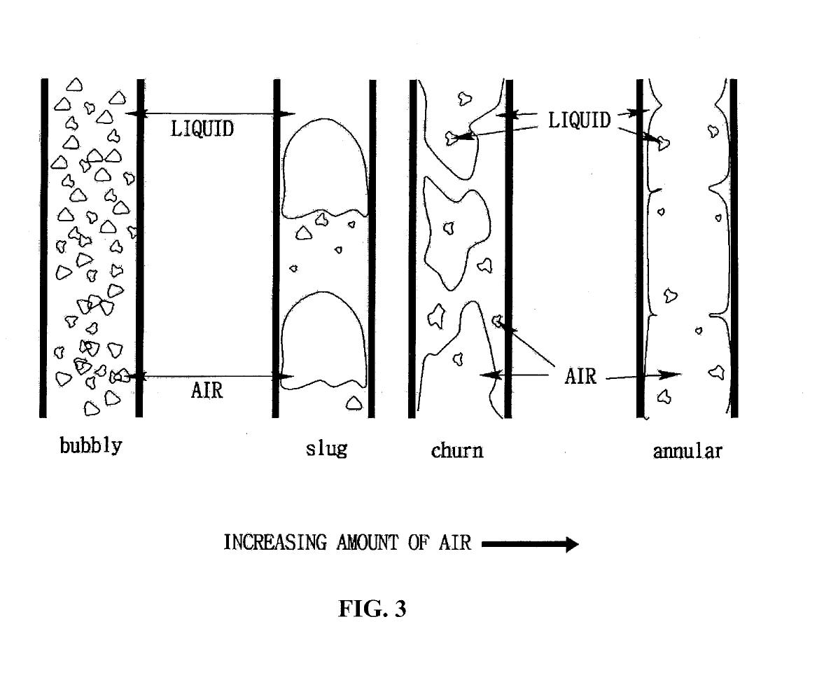

Generally, the shape of two-phase flow changes according to ε value. As shown in FIG. 3, the two-phase flow changes to bubbly, slug, churn, and annular shapes as ε value is increased. In the cylindrical tube, air bubbles have a uniformed round shape in the bubbly-shape flow and slug (or bullet) shape which almost fills the cylindrical tube in the slug-shape flow. In the churn-shape flow, most of the liquid flows in an unstable manner near the inner wall of the cylindrical tube along with the continuous flow of air bubbles of small and large sizes. In the annular-shape flow, most of the liquid flows near the wall of the cylindrical tube while air bubbles dispersed in small sizes flow near the center of the cylindrical tube.

In the present invention, ε values calculated using flux data are shown in Table 5 below. As shown in Table 5, ε values for the nozzle and the porous diffuser are similar to each other. A range of ε values of 0.2<ε<0.9 is determined to maintain constant two-phase slug flow which is effective against preventing membrane contamination.

TABLE 5
ε Value Obtained from Flux Data

| Amount of Air (Q_{g}) | Amount of Liquid (Qₗ)(L/min) | | $ɛ = \frac{{Q}_{g}}{{Q}_{g} + {Q}_{l}}$ | |
|---|---|---|---|---|
| (L/min) | Nozzle | Porous Diffu. | Nozzle | Porous Diffu. |
| 0.5 | 0.45 | 0.46 | 0.52 | 0.52 |
| 1.0 | 0.71 | 0.76 | 0.59 | 0.57 |
| 1.5 | 0.69 | 0.77 | 0.69 | 0.66 |
| 2.0 | 0.68 | 0.74 | 0.76 | 0.73 |

### Example 2

### Effect of Preventing Membrane Contamination According to Method of Introducing Air into Cylindrical Tube

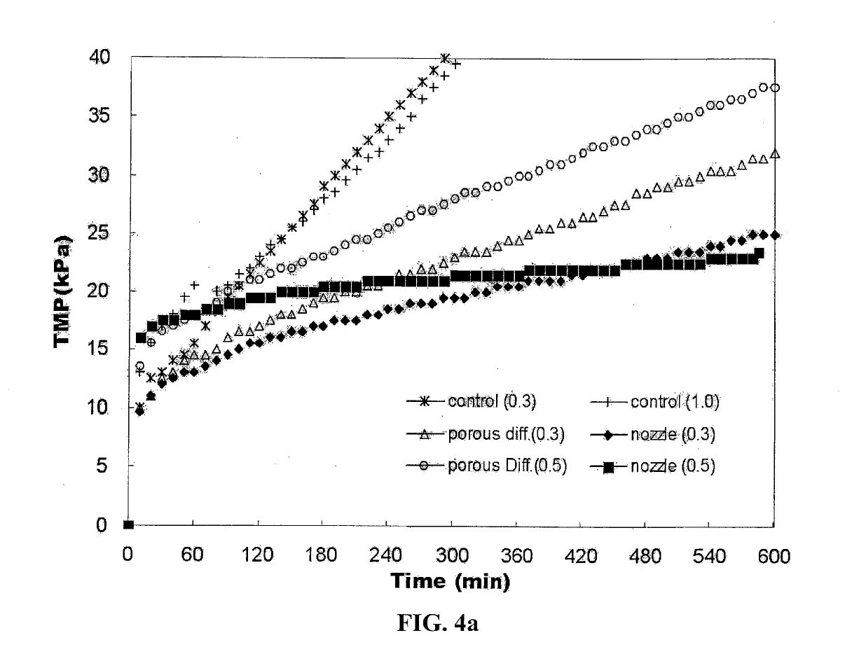

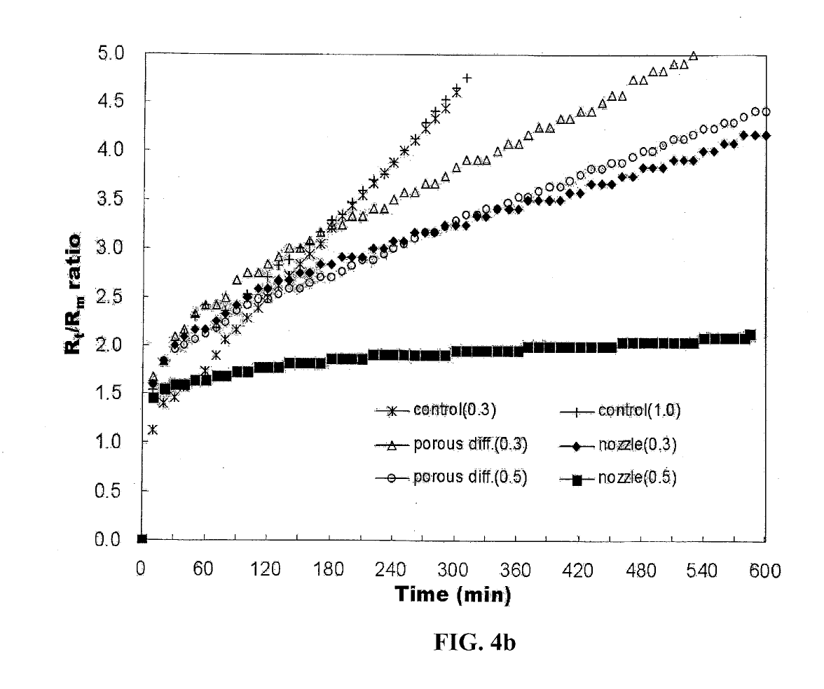

A membrane module (Run 1) having a total surface area of 0.0034 m² (10 hollow-fiber membrane strands) covered by a cylindrical tube was submerged in an MBR having an MLSS density of 6,500 mg/L. Then, the MBR was operated for 600 minutes or until TMP reached 40 kPa by maintaining a flux of 24 lm⁻¹h⁻¹. A nozzle and a porous diffuser were provided below the cylindrical tube and air was supplied at varying rates. FIG. 4A shows change in TMP with respect to time. A control group of membrane modules without the cylindrical tube was arranged on the lower end of the MBR and was operated under the same condition to determine the effect of having the cylindrical tube.

For the control group, the amount of air supplied was increased from 0.3 L/min to 1.0 L/min and the time it took to reach TMP of 40 kPa did not show a significant difference. When the amount of air supplied was increased from 0.3 L/min to 1.0 L/min, the R_{c}+R_{f} value only decreased from 4.71(10¹²×m⁻¹) to 4.56(10¹²×m⁻¹). Although the air supplied was increased by 3 times to decrease the accumulation of cake layer on the membrane surface, lessening of membrane contamination was not that significant due to air bubbles dispersing throughout the MBR instead of being concentrated around the membrane surface.

For the membrane module having provided with the cylindrical tube, the time it took to reach TMP of 40 kPa drastically increased compared with that of the control group. For example, when the air was supplied at 0.3 L/min, it took the control group 300 minutes to reach TMP of 30 kPa while it took 600 minutes for the membrane module having provided with the cylindrical tube to reach the same TMP. In comparison, when the nozzle was used, it took the membrane module having provided with the cylindrical tube 600 minutes to reach TMP of 24 kPa. Thus, the nozzle was more effective in preventing membrane contamination than the porous diffuser. Here, it is assumed that this was due to the slug-type flow, which is effective in preventing membrane contamination, induced by the air supplied by the nozzle having a diameter of 1 mm into the cylindrical tube having a diameter of 10 mm.

However, TMP increased when the amount of air supplied was increased from 0.3 L/min to 0.5 L/min from the porous diffuser. Here, it is assumed that this was due to the difference in the intrinsic membrane resistance of the membranes used in the experiments. That is, there was a slight variation in the intrinsic membrane resistance Rₘ between the membranes used in the experiments. Thus, it is difficult to accurately determine membrane contamination from the measured TMP values alone. Accordingly, in order to eliminate the effect from the intrinsic membrane resistance, Rₜ/Rₘ ratio which is shown in FIG. 4B was obtained from TMP data. As shown in FIG. 4B, when the amount of air supplied from the porous diffuser was increased from 0.3 L/min to 0.5 L/min, Rₜ/Rₘ ratio decreased, which indicated decreased membrane contamination. Thus, it was determined that the membrane module provided with the cylindrical tube is effective in preventing membrane contamination. Further, it was also determined that the nozzle is more effective than the porous diffuser in preventing membrane contamination.

### Example 3

### Contamination of Membrane Module Covered With Cylindrical Tube According to Flux

A membrane module (Run 2) having a total surface area of 0.0051 m² (15 hollow-fiber membrane strands) covered by a cylindrical tube was submerged in an MBR having an MLSS density of 6,200 mg/L. Then, the MBR was operated for 600 minutes or until TMP reached 40 kPa by changing a flux of 24 lm⁻¹h⁻¹ to 35 lm⁻¹h⁻¹, and the change in TMP with respect to time was measured. In order to eliminate the effect from the intrinsic membrane resistance, Rₜ/Rₘ ratio was obtained from the measured TMP values. The amount of air supplied was maintained at 0.3 L/min.

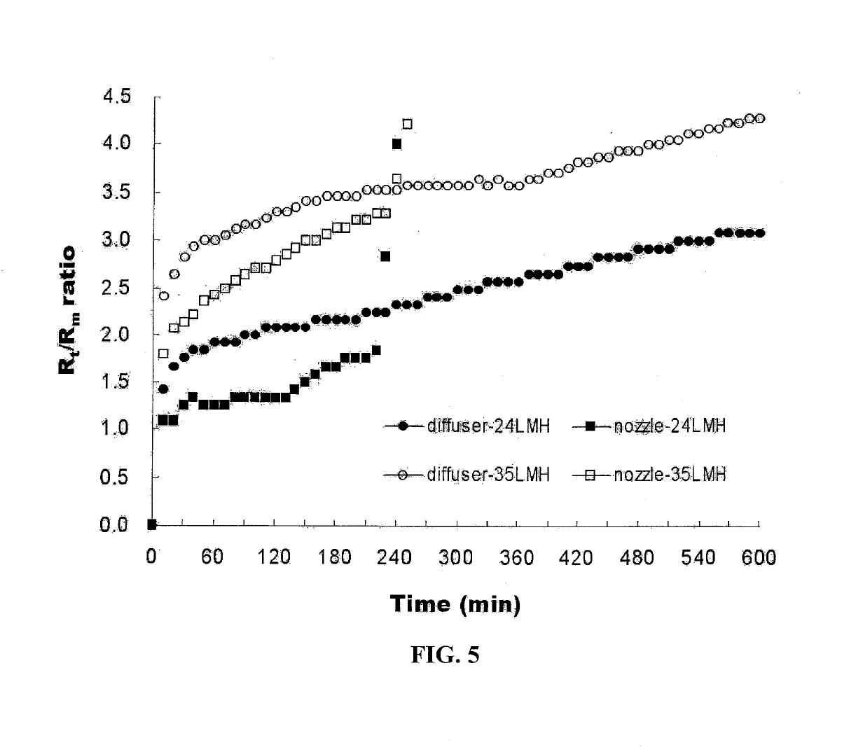

As shown in FIG. 5, Rₜ/Rₘ ratio increased less at the flux of 24 lm⁻¹h⁻¹ than at the flux of 35 lm⁻¹h⁻¹. Further, as described in Example 2, it was determined that the nozzle is more effective than the porous diffuser in preventing membrane contamination. Here, when the flux was maintained at the higher rate, the activated sludge liquid mixture attracted to the membrane surface (convection) at a higher rate to form a cake layer to increase the membrane contamination. Especially, when the MBR was operated above the critical flux, this increased membrane contamination was observed.

However, when the amount of air supplied from the nozzle was maintained at 0.3 L/min, Rₜ/Rₘ ratio increased gradually up to 220 minutes then suddenly increased thereafter. This was seen when the MBR was respectively operated at the flux of 24 lm⁻¹h⁻¹ and 35 lm⁻¹h⁻¹. At the end of the experiment, the membrane module was examined from the top of the cylindrical tube, and it was observed that the activated sludge liquid mixture has accumulated between the membrane strands to clog the cylindrical tube. However, this was not seen when the air was supplied from the porous diffuser at the same rate. Air bubbles from the porous diffuser rising in the cylindrical tube have a less tendency of being concentrated in a certain region in the cylindrical tube than the air bubbles from the nozzle by being dispersed throughout the tube. Whereas, the air bubbles from the nozzle continuously rise vertically in the center of the cylindrical tube more than near the inner wall. Accordingly, when the amount of air supplied is not enough, it is determined that the activated sludge liquid mixture accumulates on the inner wall to suddenly clog the cylindrical tube. When a sufficient amount of air is supplied from the nozzle or when sludge is not accumulated on the inner wall, it is determined that the slug-type flow induced by the nozzle is more effective in controlling the membrane contamination than the porous diffuser. This is also seen in the results from the previous experiments described above.

In the case when the nozzle is used, the accumulation of sludge may be due to the volume of occupied by the membrane in the cylindrical tube. That is, the volume occupied by the membrane increases as more membrane strands having the same length are used, which increases the cross-sectional area Aₘ occupied by the membrane in the cylindrical tube. Thus, the cross-sectional area ratio Aₘ/Aₜ of the membrane to the cylindrical tube increases as more membranes strands are used. Here, the membrane surface scouring effect of the air bubbles from the nozzle which rises vertically in the slug-type flow may be affected by the Aₘ/Aₜ ratio. As such, there is a need to analyze the effect the cross-sectional area ratio of the membrane to the cylindrical tube has on preventing membrane contamination, which is explained in the next example.

### Example 4

### Membrane Contamination Preventing Effect by Volume Occupied by Membrane Module in Cylindrical Tube

Membrane modules respectively having 10 membrane strands for Run 1, 15 membrane strands for Run 2, 30 membrane strands for Run 3, were respectively covered with a cylindrical tube. While supplying air at a rate of 0.5 L/min and maintaining a flux of 24 lm⁻¹h⁻¹ to 35 lm⁻¹, TMP was measured, which was then used to calculate Aₘ/Aₜ ratio.

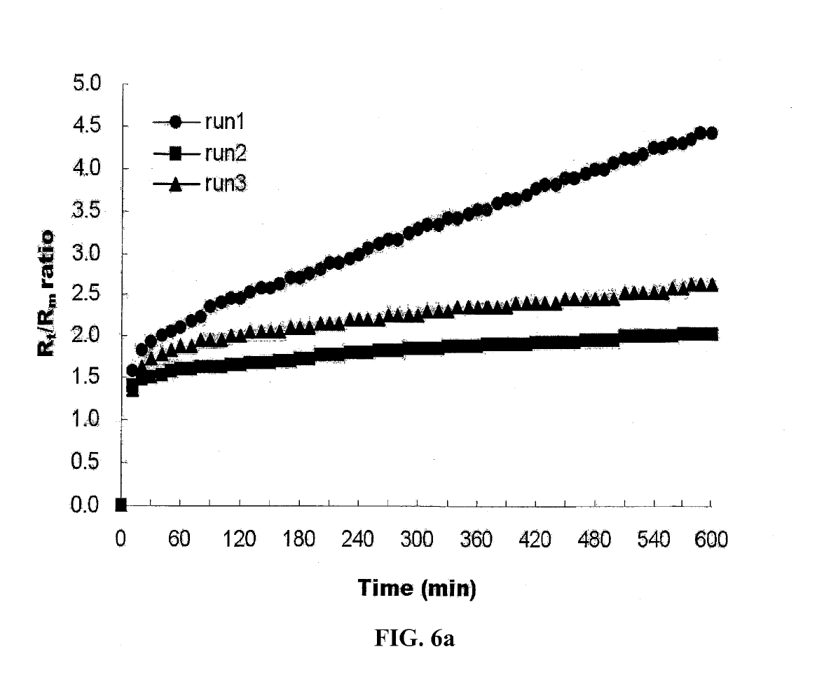

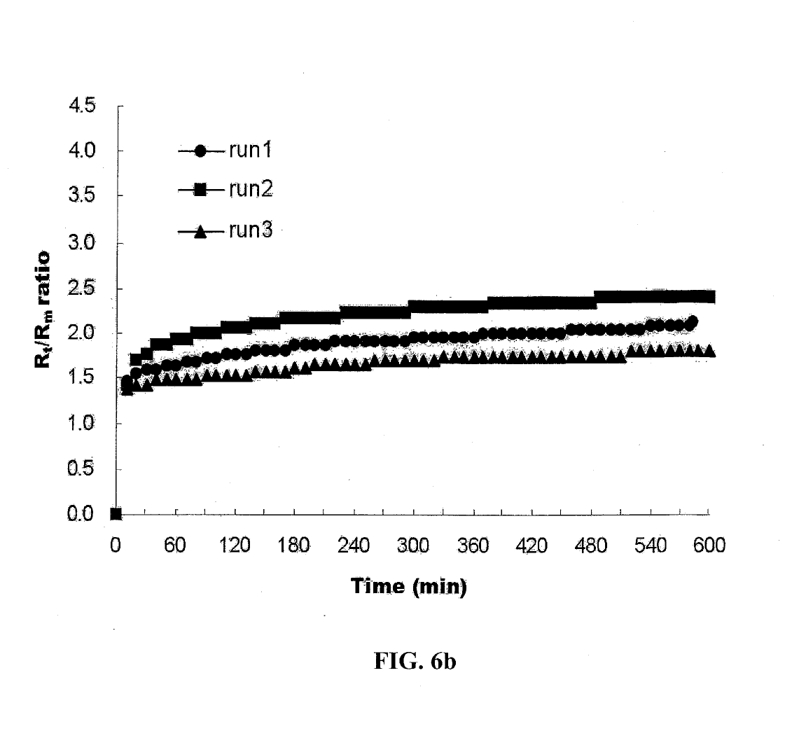

As shown in FIG. 6A, when a porous diffuser was used to supply air, Rₜ/Rₘ ratio rapidly increased in Run 1 and increased the least in Run 2. That is, the membrane cleaning effect by air bubbles was the lowest in Run 1, which had the lowest cross-sectional area occupied by the membrane strands in the cylindrical tube—with the Aₘ/Aₜ ratio of 0.18. Here, due to the small cross-sectional area occupied by the membrane in the cylindrical tube, most of the air bubbles dissipated throughout the empty space in the cylindrical tube before making it to the membrane strands to cause the rapid rise in the resistance. In Run 2, Aₘ/Aₜ ratio was increased from 0.18 to 0.27 and, after 600 minutes, Rₜ/Rₘ ratio decreased by 54% from that of Run 1. Here, due to the increased cross-sectional area occupied by the membrane strands in the cylindrical tube, the amount of air bubbles making contact with the membrane surface increased. However, when Aₘ/Aₜ ratio was increased from 0.27 to 0.55 in Run 3, Rₜ/Rₘ ratio increased by 30% from that of Run 2 after 600 minutes. Here, this may have been due to filling of the cylindrical tube by the increased number of membrane strands, which may have hindered the effective passing of the air bubbles in the cylindrical tube to decrease the membrane cleaning effect, thus increasing the resistance. Rₜ/Rₘ ratios for Run 1, Run 2 and Run 3 are shown in Table 6 below.

TABLE 6
Rₜ/Rₘ Ratios for Run 1, Run 2 and Run 3.

| | | Unit | Rₜ/Rₘ Ratio | | |
|---|---|---|---|---|---|
| | | Aₘ/Aₜ | 0.18 | 0.27 | 0.55 |
| | | Flux (lm⁻¹h⁻¹) | 24 | | |
| Air supply method | | Run | Run 2 | Run 2 | Run 3 |
| Porous | Air bubble | 0.3 | 5.33 | 1.99 | — |
| Diffuser | amount | 0.5 | 4.41 | 2.03 | 2.63 |
| | (L/min) | 1.0 | — | 2.65 | 4.60 |
| | | 1.5 | — | — | 2.83 |
| Nozzle | Air bubble | 0.3 | 4.17 | 1.84 | — |
| | amount | 0.5 | 2.14 | 2.41 | 1.81 |
| | (L/min) | 1.0 | — | 2.59 | 2.33 |
| | | 1.5 | — | — | 2.54 |

In comparison, when a nozzle was used to supply air, Rₜ/Rₘ ratio rapidly increased in Run 2 and increased the least in Run 3, as shown in FIG. 6B. In Run 2, Aₘ/Aₜ ratio was increased from 0.18 to 0.27 and, after 600 minutes, Rₜ/Rₘ ratio increased by about 13% from that of Run 1, opposite to decreased Rₜ/Rₘ ratio when the porous diffuser was used under the same conditions. When Aₘ/Aₜ ratio was increased in Run 3, Rₜ/Rₘ ratio decreased by 25% from that of Run 2 to exhibit the lowest Rₜ/Rₘ ratio. Here, this may have been due to rising of the air bubbles in a slug-shape flow in and between the membrane strands. While in Run 1, the membrane contamination may have been decreased due to the small cross-sectional area of the membrane strands which allowed a slug-type flow to be formed in a space between the inner wall of the cylindrical tube and the membrane strands, the slug-type flow in Run 2 may not have been formed due to the little space available between the inner wall of the cylindrical tube and the membrane strands which occupied more space within the cylindrical tube with their large cross-sectional area. However, in Run 3, since the space between the membrane strands and the inner wall of the cylindrical tube are even smaller due to the further increased cross-sectional area of the membrane strands, the air bubbles may have flowed between the membrane strands in a slug-type flow to decrease the membrane contamination.

To conclude the results from the above, it was determined that the increase or decrease in membrane contamination was not due to increased or decreased Aₘ/Aₜ ratio, rather there is an optimum Aₘ/Aₜ ratio which renders minimum membrane contamination. When the porous diffuser is used in Run 2 carried out with 20 membrane strands, minimum membrane contamination was observed. Similarly, when the nozzle was used in Run 3 carried out with 30 membrane strands, minimum membrane contamination was also observed. The effect of decreasing membrane contamination by air bubbles depends largely on Aₘ/Aₜ ratio, and it was determined that the optimum Aₘ/Aₜ ratio for the case when the nozzle was used is different from the case when the porous diffuser was used.

As described above, the present invention provides a submerged MBR including a membrane module covered with a cylindrical tube on its outer circumference to decrease the resistance of a cake layer which causes membrane contamination and decreases flux during the operation of the submerged MBR. The cylindrical tube covering the membrane module prevents the air supplied from a nozzle or a porous diffuser from escaping from the vicinity of the membrane module to thus maximize the cleaning effect of air bubbles have on membrane strands provided in the membrane module.

By using both the nozzle and the porous diffuser, different methods of supplying air were implemented to observe the presence of two-phase flow of liquid and air bubbles with respect to change in the amount of air supplied. Further, the number of membrane strands in the membrane module and flux were changed to observe the effect this has on decreasing membrane contamination. As a result, the submerged MBR of the present invention has one or more of the following performances, effects, and advantages.

The MBR having implemented with a nozzle is more effective in preventing membrane contamination than using a porous diffuser when the same amount of air is supplied. Here, it was determined that air bubbles generated by the nozzle rise inside the cylindrical tube in a slug type two-phase flow of liquid and air bubbles which is effective in decreasing membrane contamination.

However, when an insufficient amount of air is supplied from the nozzle, activated sludge liquid mixture quickly accumulated on the wall of the cylindrical tube to cause a rapid clogging of the cylindrical tube, and, after a certain period time, the membrane strands contaminated more suddenly than when the air is supplied from the porous diffuser. Here, it was determined that the increase or decrease in membrane contamination was not due to increased or decreased Aₘ/Aₜ ratio, rather there is an optimum Aₘ/Aₜ ratio which renders minimum membrane contamination.

That is, when the submerged MBR according to the present invention is implemented with a porous diffuser as the air supply means, the optimum Aₘ/Aₜ ratio, which renders a maximum effect in decreasing membrane contamination, is determined to be about 0.25 to about 0.30, and when implemented with a nozzle as the air supply means, the optimum Aₘ/Aₜ ratio is determined to be about 0.50 to about 0.60.

While the present invention has been particularly shown and described with reference to exemplary embodiments thereof, it will be understood by those of ordinary skill in the art that various changes in form and detail may be made therein without departing from the spirit and scope of the present invention as defined by the following claims. The exemplary embodiments should be considered in a descriptive sense only and not for purposes of limitation.

What is claimed is:

1. A submerged membrane bio-reactor, comprising:
a submerged membrane module having a membrane with a hollow fiber structure;
a cylindrical tube covering an outer circumference of the submerged membrane module; and
a nozzle provided within a treatment vessel to supply air to inside of the cylindrical tube.

2. The submerged membrane bio-reactor of claim 1, wherein the nozzle has a slender tube shape and is provided at a lower side of the cylindrical tube.

3. The submerged membrane bio-reactor of claim 1, wherein the nozzle has a diameter of from about 0.1 mm to about 10 mm.

4. The submerged membrane bio-reactor of claim 1, further comprising a porous diffuser provided within a treatment vessel to supply air to inside of the cylindrical tube,
wherein a cross-sectional area ratio (Aₘ/Aₜ) of the membrane to the cylindrical tube is from about 0.25 to about 0.30, with Aₘ being a cross-sectional area of the membrane in the submerged membrane module, and Aₜ being a cross-sectional area of the cylindrical tube.

5. The submerged membrane bio-reactor of claim 1, wherein the nozzle comprises a cross-sectional area ratio (Aₘ/Aₜ) of the membrane to the cylindrical tube from about 0.50 to about 0.60, with Aₘ being a cross-sectional area of the membrane of the submerged membrane module, and Aₜ being a cross-sectional area of the cylindrical tube.

6. The submerged membrane bio-reactor of claim 1, wherein the cylindrical tube has a cylindrical body and a conical lower portion formed on the cylindrical body, the conical lower portion having a gradually increasing inside diameter larger than that of the cylindrical body.
